(19) United States
(12) Patent Application Publication
(10) Pub. No.: US 2026/0047957 A1
(43) Pub. Date: Feb. 19, 2026

(54) INSTRUMENT FOR POSTPARTUM PLACEMENT OF INTRA-UTERINE DEVICE AND PROCESS THEREOF

(71) Applicant: Pregna International Limited, Mumbai (IN)

(72) Inventors: Paul D Blumenthal, Palo Alto, CA (US); Ajit Raje, Mumbai (IN); Mukul Taparia, Mumbai (IN)

(21) Appl. No.: 19/220,167

(22) Filed: May 28, 2025

(30) Foreign Application Priority Data
Aug. 14, 2024 (IN) 202421061624

(51) Int. Cl. A61F 6/18

(52) U.S. Cl. CPC A61F 6/18

(57) ABSTRACT

An instrument (110) for postpartum placement of IUD (30), comprising a placement tube (114) of an extended length (120) having a rotation constraining means at a fundal end (118), a pair of guiding slots (142A) at a gripping end (117), a referencing mark or projection (144) at the gripping end (117) in a defined position with respect to the rotation constraining means; a placement string (121) disposed removably on the IUD (13); a string retainer (131) and a string lock (132); a placement length of the placement string (121) includes a portion for holding a first end (121A) of the placement string (121) while a second end (121B) is trapped by the string retainer (131) while the IUD (130) is situated at the fundal end (118) of the placement tube (114) with the placement string (121) taut and arms (129) of the IUD (30) constrained against rotation.

### FIELD OF THE INVENTION

The present invention relates to an instrument to insert intra-uterine device (abbreviated as IUD) in uterus and the process thereof, soon after delivery, medically known as postpartum insertion of IUD.

### BACKGROUND OF THE INVENTION

Studies on postpartum implantation of intra-uterine devices approve placing intra-uterine device (IUD) shortly after childbirth; as the vagina, cervix and uterus are enlarged, which eases emplacement. While visible advantage of Postpartum insertion of IUD is that it is painless, does not cause additional bleeding and does not interfere with lactation, another socially and economically important purpose of the postpartum insertion is that, as the IUD is placed immediately after delivery, the woman does not need to make effort to visit hospital/nursing home again for getting IUD inserted. This is because women in several developing countries are not pro-active in deploying the contraception methods.

In the past, many concepts have been made related to postpartum IUD insertion. A prior art document describes inserter for placing IUD in the uterine cavity in the immediate post-partum period. This device comprises of two cylindrical rods-outer and inner, engaged with each other with the help of locking member. A needle is attached to the IUD. This needle helps in attachment of IUD to uterine cavity. This process essentially thrusts the needle by pushing it using inner rod and can possibly result into excessive puncture of fundus.

U.S. Pat No. 3,954,103 discloses an inserter for placing intra uterine device in uterus after the immediate childbirth manually; where there is need for the doctor to put hand inside the cavity and fix the apparatus with needle. This method of placing IUD is unhygienic and high skill based, therefore hazardous.

U.S. Pat No. 5,303,717 describes a device for fixing an intra uterine device in uterus comprising of a non-biodegradable thread attached to contraceptive device, connected to the retaining member, made up of biodegradable material which helps in attachment to the post-partum state of uterus, and which slowly gets dissolved after uterus returns to normal state. This method also uses needle to affix IUD in uterus making it high risk and painful.

Patent No. FR2538243A1 discloses a cannula or tubular sheath inside which can move a piston also provided with a nylon thread, fixed at the upper end and free at the lower end. In practice, the IUD loop is hooked to the knot formed by the plunger wire, and the IUD in the hollow upper part of the cannula which we introduce into the vaginal canal, we then push the piston upwards to release the device and apply it to the uterine cavity. Then, by gently pulling the plunger, separate it from the IUD loop that stays outside the cervical opening. The presence of this loop also naturally makes it easier to remove the IUD when this operation becomes necessary.

Patent No. WO1980000536 discloses a marking system by centimetric coloring of the thread(s) attached to all types of IUD, which makes it a permanent measuring instrument and allows to know with precision the place of the IUD in the uterus. U.S. Pat No. 4,721,105 discloses a thread (10) affixable to the IUD (9; 40) and affixed to a retaining member (10′; 14; 15; 19), the retaining member (10″; 14; 15; 19) loosely engages the needle (2; 26; 29; 44), means (4; 22; 30; 49) are provided for achieving and maintaining a traction on the thread (10), said traction means adapted to ensure the cooperation of the retaining member (10″; 14; 15; 19) with the needle (2; 26; 29; 44) as long as the means (5,6,7; 25,28; 35,36; 48,49) for locking the actuating member with regard to the protecting member are not dis engaged and the IUD is not released.

It is common and therefore well known that many surgeons use Kelly clips or placental forceps as an apparatus for postpartum insertion, since the size of uterus is adequately large even to receive large handles of Kelly clips. This process requires sterilization every time. Also, there is possibility of IUD getting dropped inappropriately. Moreover, there is possibility of these surgical instruments causing possible damage to the tender body parts by inadvertent slip or hand movement.

India Patent No. 446341 discloses an instrument for postpartum insertion of intra-uterine device maintaining a specific orientation while insertion. An extra-long string makes the insertion process possible.

The prior art methods and the apparatus for inserting IUD immediately after childbirth have been found to leave significant scope of improvement towards a satisfactory degree of ease, particularly considering no necessity to subsequently visit delivery center or clinic.

Our Invention Overcomes These Problems.

### OBJECTIVE OF THE INVENTION

The objective of the invention is to provide an instrument to insert the intra-uterine device (IUD) in the postpartum condition of uterus, hygienically.

Another objective is to invent a simple instrument to insert, in specific orientation the intra-uterine device (IUD) in the postpartum condition of uterus.

Yet another objective of present invention is to provide an IUD inserter which can be used soon after childbirth thus not requiring woman's additional visit for implanting intra-uterine device (IUD).

Yet another objective of the invention is to provide an intrauterine device which is as hygienic, safe and proven for inserting in the uterus of the female as current devices.

Yet another objective of the invention is to provide an intrauterine device which is removable with minimum trauma.

Yet another objective of the invention is to provide an instrument that is easy to use by medical service provider for insertion.

### SUMMARY OF INVENTION

An instrument according to present invention comprises a placement tube of an extended length carrying an IUD having a retrieval string, at its fundal end and having a string retainer and a string lock. The instrument is packaged in a transparent pouch, sterilized by gamma irradiation.

The placement tube has a rotation constraining means, wherein in the preferred embodiment the rotation constraining means is a pair of constraining slots at a fundal end of the placement tube which ensures that the IUD maintains a specific orientation. Another corresponding pair of guiding slots is provided at a gripping end of the placement tube. Both pairs of slots have rounded edges and a converging opening, which facilitate injury-less insertion of the placement tube in the uterus in a safe and correct specific orientation. A projection at the gripping end, provided in defined relation with the slots, and which is preferably in-between the pair of slots, makes it possible to ascertain a specific orientation of IUD from the gripping end.

The extended length of the placement tube corresponds to a size of the postpartum uterus, which is several times bigger than a normal uterus. A placement tube is accordingly at least twice longer than corresponding part of any known inserter for insertion in normal sized uterus. The extended length includes a sufficient portion of length for holding the placement tube fully and firmly, and for that a gripping end of the placement tube must project sufficiently out of vagina, through postpartum uterus and dilated cervix when a fundal end of the placement tube is in proximity of fundus. The placement tube is made of a thermoplastic material with material property and a thickness such that it can axially bend slightly and take required curvature while insertion, as needed by the shape of uterus.

While the placement tube is therefore flexible, it is of the extended length which is significantly more than what is suitable for a normal uterus. Thus, it is important that it is not unduly flexible/sagging and has rigidity enough so as to carry the IUD till the fundus, without possibility of dropping the IUD midway. Also, the placement tube should not buckle under the back pressure as may be exerted due to resistance from cervix or uterine wall. A thickness of the placement tube therefore has to be accordingly optimum and in relation to the material property of selected thermoplastic. Typically, the thickness of walls of the placement tube is of the order of 0.5 to 2.5 mm for high density polyethylene, polypropylene, or equivalent material. A diameter of the placement tube is NOT constrained by cervix since cervix of post-partum uterus is several times wider than a normal cervix and a diameter of 5 mm to 8 mm is optimum mechanically, medically as well as economically.

The IUD comprises an IUD frame and a non-removable retrieval string.

A placement string is disposed removably on the IUD. A first end of the placement string is slid into an orifice in a bulge of the IUD frame. The first end along with a second end forms a placement length such that, with the IUD residing at the fundal end of the placement tube, a manually holdable portion of the placement string including the first end and the second end projects out of the gripping end of the extended length of the placement tube.

The retrieval string unremovably disposed from the bulge of the IUD is knotted around the orifice of the bulge in the middle of the retrieval string such that a hanging length including both the hanging ends is of 4 cm to 13 cm. Such a length generally is sufficient to emerge out of cervical os of a normal uterus, that is, a uterus which has regained its near original size after a few weeks of child birth.

The retrieval string permanently disposed or originating from the bulge of the IUD is generally integrally molded along with the IUD frame such that a hanging length including the hanging end is at least 4 cm to 13 cm.

The retrieval string which is permanently disposed or originating from the bulge is either of uniform circular cross-section, or of gradually reducing cross section or of reducing cross section in one or more steps, such that the retrieval string remains more or less straight and does NOT curl. This aspect shall be further elaborated.

A string retainer is a cuboid or a cylindroid shaped non-hard component with an axial through-hole via which the string retainer slides on and tight-fits on the placement tube. Once mounted on the placement tube, it stay puts linearly as well as rotatingly, that is, it does not rotate nor slide freely. A reference mark is provided on one of its flat faces to ascertain its angular position w.r.t. any mark or projection on the placement tube.

A string lock is a circular non-hard component with a concentric hole via which the string lock slides on and tight-fits on the placement tube. Once mounted on the placement tube, it stay puts linearly as well as rotatingly, that is, it does not rotate nor slide freely.

The instrument is assembled by firstly inserting the first end and the second end of the placement string of the IUD from the fundal end of the placement tube, and continuing so with the hanging end(s) of the retrieval strings also inserted, and at this point the first end and the second end of the placement string would emerge out of the gripping end of the placement tube. The first end and the second end is then pulled ensuring that the arms of the IUD sit in the two respective slots of the placement tube. Keeping both the ends of the placement string taut, the string retainer is then slid on the placement tube slipping over the placement string, till the string retainer carries the placement string along and is close to the two ends of the placement string; and the ends of placement string get trapped and constrained consequently. Finally the string lock is slid on the placement tube from the gripping end and positioned just after the gripping end of the placement tube. The placement string in the vicinity of the first end and the second end is ensured to be sitting in the respective slot at the gripping end while the placement string is ensured to be taut, such that the arms of the IUD sit firmly in the slots and cannot get dislodged from the slots inadvertently and unless the placement string is loosened consciously or unlocked from string lock as well as string retainer.

For process of inserting, the instrument is taken out of the transparent pouch by holding the placement tube from the gripping end. The instrument is inserted into uterus from the fundal end, by continuing to hold the placement tube with one palm. Due to large opening of the dilated vagina, cervix and uterus, the overall size of the IUD, and particularly a width of the IUD is insignificant, which is otherwise of high significance in normal i.e. non-postpartum situation. The IUD is carried inside the enlarged uterine cavity till touching of the IUD is sensed by surgeon at the fundus of uterus. The specific orientation of the IUD is maintained by keeping an eye on the projection and or the string retainer.

The uterine cavity is felt externally by another palm, pushed down externally with palm and the IUD is constrained using any significant finger on the abdomen. Keeping the IUD constrained externally as thus described, at this moment, the placement string is freed at the gripping end of the placement tube by dismounting the string lock from the gripping end. The placement string is cut away by a scissors at a vicinity of the second end such that a part of the placement string in the second vicinity of the second end remains gripped by the string retainer. The placement string is pulled out from a vicinity of the first end till the entire remaining placement string gets pulled out of the placement tube, including a cut end, created due to cutting of the placement string, coming out of the hole of the bulge; and the placement tube is then withdrawn from the uterus gradually and safely.

It is important to note that as the postpartum uterus shrinks gradually, the retrieval string has a tendency to curl and get collected in the uterus randomly. While the function of retrieval or removing is still easily performed by a service provider by pulling the retrieval string out through the cervix, the self-assurance function is lost. It is due to this reason that the present invention, as a variation, provides for a string of higher rigidity for a part of the retrieval string so that in the course of uterus gradually shrinking, the natural funnel shaped uterus converging into cervix acts as a mechanical guide and the retrieval string of enhanced rigidity eventually emerges out of the cervical os, as it happens during IUD placement in a normal uterus.

In above description and corresponding Figures, IUD frame of device (IUD) of commonest shape “T” is shown, however, the instrument and process is suitable for different intra-uterine devices of other shapes. For IUDs of shape other than a “T” frame, any part of construction that prevents rotation of IUD is accordingly constrained by the appropriate constraining means.

A low cost quick instrument is possible as a variation and a quick make shift arrangement.

### BRIEF DESCRIPTION OF DRAWINGS

FIG. 1 is a perspective view of an instrument as per present invention.

FIG. 2 is a partial perspective view of an intra-uterine device (IUD) parked at a fundal end of a placement tube.

FIG. 3 is a perspective view of the placement tube.

FIG. 4 is a partial side view of the placement tube.

FIG. 5 is a partial perspective view of a gripping end of the instrument.

FIG. 6 is a perspective view of the IUD.

FIG. 7 is a perspective view of the IUD with a placement string.

FIG. 8 is a perspective view of a string retainer while FIG. 9 is a perspective view of a string lock.

FIG. 10 is a representative front view of the placement tube illustrating flexibility in apposition to uterus.

FIG. 11 is a front view of the instrument in a transparent pouch.

FIG. 12 is a representative sectional view of a postpartum uterus with the instrument inserted.

FIG. 13 is a representative sectional view of the postpartum uterus with the string lock of the instrument dismounted.

FIG. 14 is a representative sectional view of postpartum uterus with a placement string being cut.

FIG. 15 is a representative partial side sectional view of postpartum uterus of a woman with the instrument inserted.

FIG. 16 is a representative sectional view of postpartum uterus with an end of the placement string pulled out from the string retainer.

FIG. 17 is a representative sectional view of postpartum uterus with the placement string being pulled out entirely.

FIG. 18 is a representative sectional view of a normal uterus with a curled up retrieval string.

FIG. 19 is a front view of IUDs with embodiments of permanently disposed or originating retrieval strings.

FIG. 20 is a representative front view of IUDs with different shapes analogues to T shape IUDs.

FIGS. 21 and 22 are perspective views of an add-on carrier, and a corresponding low cost quick instrument, as another variation.

### DETAILED DESCRIPTION OF INVENTION

Preferred embodiment of this invented instrument for postpartum insertion and removal of intra-uterine device will now be described in detail, with reference to the accompanying drawings. The terms and expressions which have been used here are merely for description and not for limitation.

Placement of the IUD in post-partum uterus in the safe and correct specific orientation of the IUD needs a different understanding than placement in normal uterus. A front inner skin surface (147) and a rear inner skin surface (148) of normal uterus is generally in close proximity, between which any IUD frame (128) inserted shall normally get sandwiched flatly, that is, the entire frame of IUD touching both inner skins. In other words, the IUD in a normal uterus is effortlessly placed in a correct specific orientation due to natural room in uterus. However, in a post-partum uterus, a huge empty volume in the uterus, separating the front inner skin surface (147) and the rear inner skin surface (148), caused after child birth has a possibility of IUD frame (128) occupying random position including IUD frame (128) resting on an edge of the arm, and such random placement may cause a puncture or injury in the uterus walls as the uterus shrinks gradually! The present invention is focused on placing the IUD frame (128) flatly on the rear inner skin (148) so that the IUD gets sandwiched in the same manner as in a normal uterus, as the uterus substantially regains its normal shape, and prevents angular placement resulting into aggravated placement with potential to harm the inner skin surfaces of uterus.

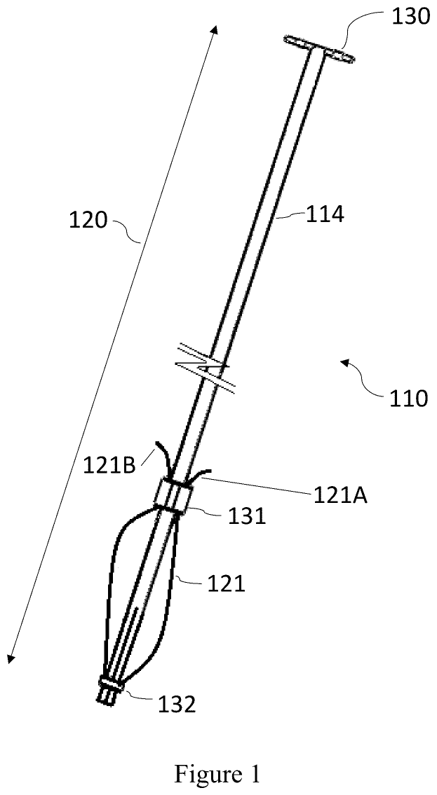

FIG. 1, an instrument (110) according to present invention comprises a placement tube (114) of an extended length (120) carrying an IUD (130) with a non-removably disposed retrieval string (115) and a placement string (121) removably slipped on, having a string retainer (131) and a string lock (132), the IUD (130) parked in the placement tube (114) at its fundal end (118). The instrument (110) is packaged in a transparent pouch (111), sterilized by gamma irradiation.

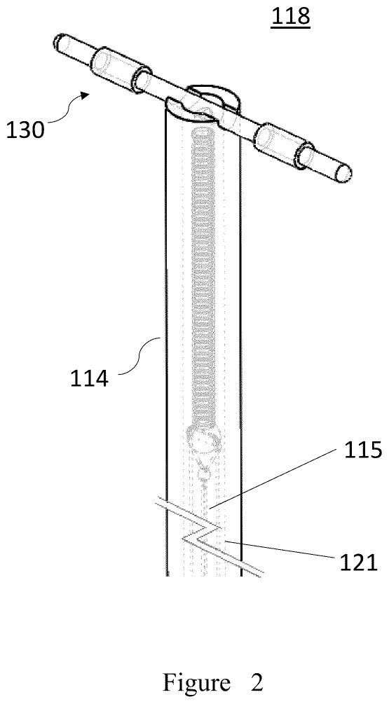

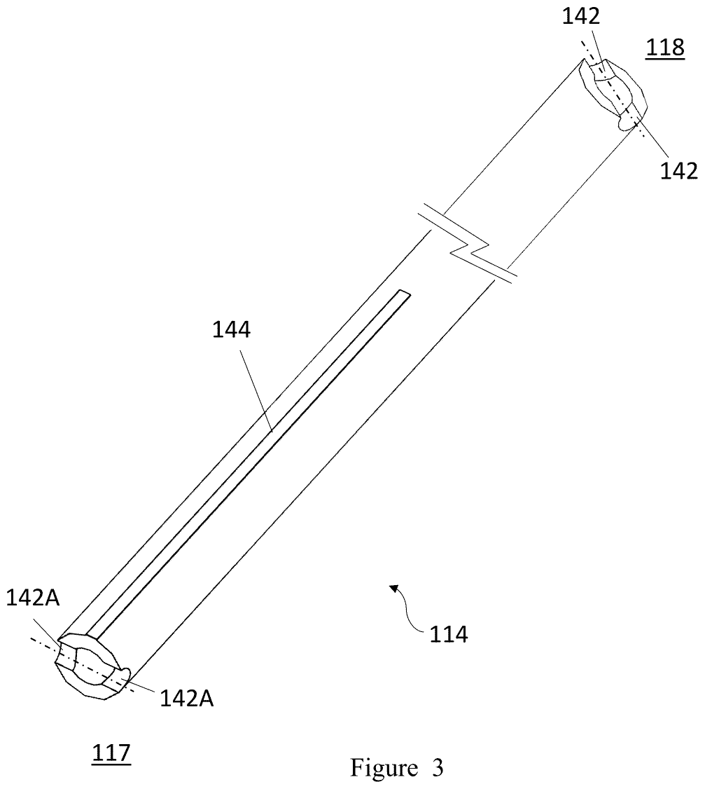

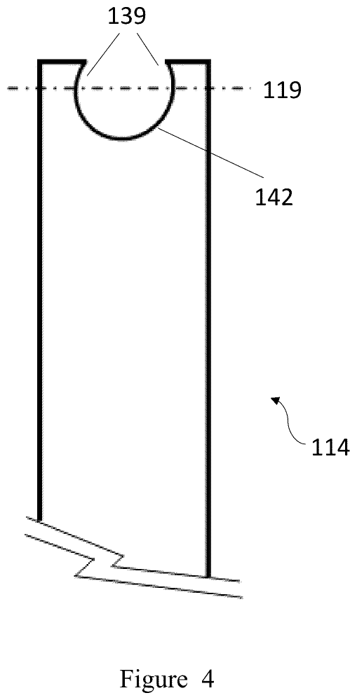

FIGS. 2, 3, 4, the placement tube (114) has a rotation constraining means, wherein in the preferred embodiment the rotation constraining means is a pair of constraining slots (142) integrally formed at a fundal end (118) of the placement tube (114). The constraining slots (142) have a slot radius (122) slightly larger than an arm radius of arms (129) of the IUD (130), which facilitates the IUD (130) maintaining a specific orientation during placement in uterus. Another corresponding pair of guiding slots (142A) is provided at a gripping end (117) of the placement tube (114). The pair of guiding slots (142A) is axially parallel to the pair of constraining slots (142). Both pairs of slots (142, 142A) have rounded edges and comprise an arch larger than a semi-circle, resulting into a converging opening (139), which facilitate injury-less insertion of the placement tube (114) in the uterus (150) in a safe and correct specific orientation. A projection (144) at the gripping end (117), provided in defined relation with the pair of constraining slots (142), and which is preferably in-between the pair of slots (142, 142A), makes it possible to ascertain a specific orientation of IUD (130) from the gripping end (117).

The extended length (120) of the placement tube (114) corresponds to a size of the postpartum uterus, which is several times bigger than a normal uterus. A placement tube (114) is accordingly at least twice longer than corresponding part of any known inserter for insertion in normal sized uterus. The extended length (120) includes a sufficient portion of length for holding the placement tube (114) fully and firmly, and for that a gripping end (117) of the placement tube (114) must project sufficiently out of vagina, through postpartum uterus (150) and dilated cervix when a fundal end (118) of the placement tube (114) is in proximity of fundus (149).

The placement tube (114) is made of a thermoplastic material with material property and a thickness (119) such that it can axially bend slightly and take required curvature (116) while insertion, as needed by the shape of uterus (150).

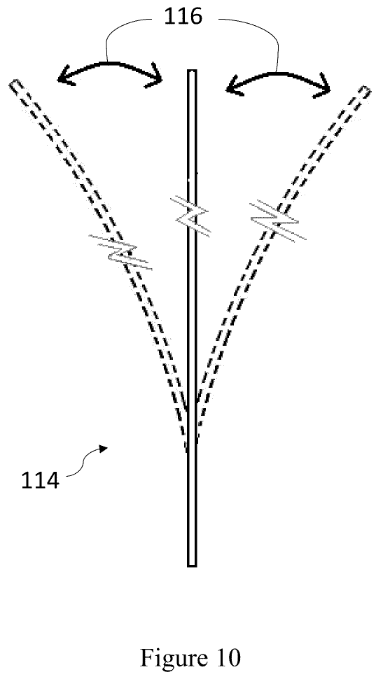

While the placement tube (114) is therefore flexible, FIG. 10; it is of the length (120) which is significantly more than what is suitable for a normal uterus (150). Thus, it is important that it is not unduly flexible/sagging and has rigidity enough so as to carry the IUD (130) till the fundus (149), without possibility of dropping the IUD (130) midway. Also, the placement tube (114) should not buckle under the back pressure as may be exerted due to resistance from cervix or uterine wall. A thickness (119) of the placement tube (114) therefore has to be accordingly optimum and in relation to the material property of selected thermoplastic. Typically, the thickness (119) of walls of the placement tube (114) is of the order of 0.5 mm to 2.5 mm for high density polyethylene, polypropylene, or equivalent material. A diameter of the placement tube (114) is NOT constrained by cervix since cervix of post-partum uterus is several times wider than a normal cervix and a diameter of 5 mm to 8 mm is optimum mechanically, medically as well as economically.

In other words, flexibility and rigidity optimization is an important aspect of the placement tube (114) due to the postpartum shape and size of uterus (150) so that the placement tube (114) rigidly carries the IUD (130) till the fundus (149).

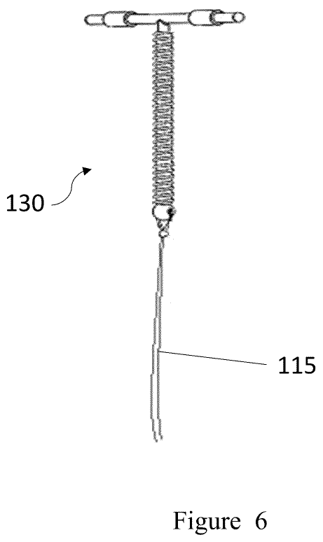

FIG. 6, the IUD (130) comprises a known IUD frame (128) provided with a non-removable retrieval string (115).

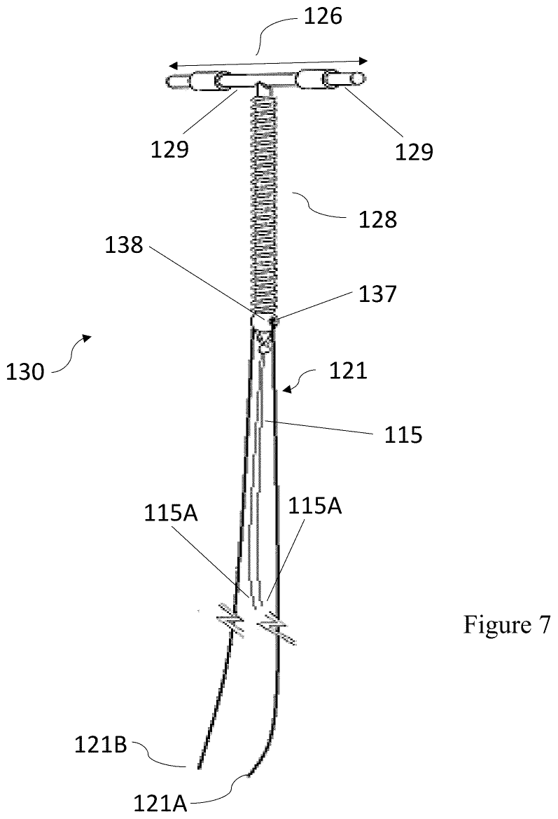

FIG. 7, the placement string (121) is removably disposed on the IUD frame (128). A first end (121A) of the placement string (121) is slid into an orifice (137) in a bulge (138) of the IUD frame (128) of the IUD (130). The first end (121A) along with a second end (121B) till the bulge (138) forms a placement length such that, with the IUD (130) residing at the fundal end (118) of the placement tube (114), a manually holdable portion of the placement string (121) including the first end (121A) and the second end (121B) projects out of the gripping end (117) of the extended length (120) of the placement tube (114). The placement length therefore also implies including a portion of placement string length for holding a first end (121A) of the placement string (121) while a second end (121B) is trapped by the string retainer (131) while the IUD (130) is situated at the fundal end (118) of the placement tube (114) with the placement string (121) taut and arms (129) of the IUD (30) constrained against rotation by the rotation constraining means.

The retrieval string (115) unremovably disposed from the bulge (138) of the IUD (130) is knotted around an orifice (137) of the bulge (138) in the middle of the retrieval string (115) such that a hanging length (115B) including both the hanging ends (115A) is of 4 cm to 13 cm. Such a length generally is sufficient to emerge out of cervical os of a normal uterus, that is, a uterus which has regained its near original size after a few weeks of child birth.

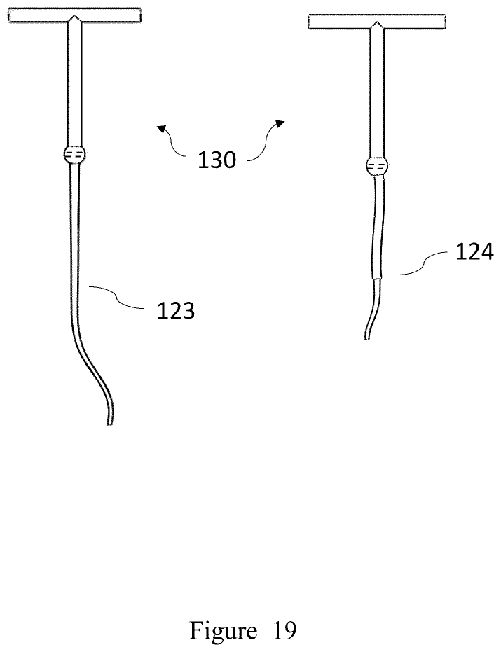

FIG. 19, as a variation, the retrieval string (115) permanently disposed or originating from the bulge (138) of the IUD (130) is generally integrally molded along with the IUD frame (128) such that a hanging length (115B) including the hanging end (115A) is at least 4 cm to 13 cm. The retrieval string (115) which is permanently disposed or originating from the bulge (138) is either of uniform circular cross-section, or of gradually reducing cross section (123) or of reducing cross section in one or more steps (124), such that the retrieval string remains more or less straight and does NOT curl. This aspect shall be further elaborated.

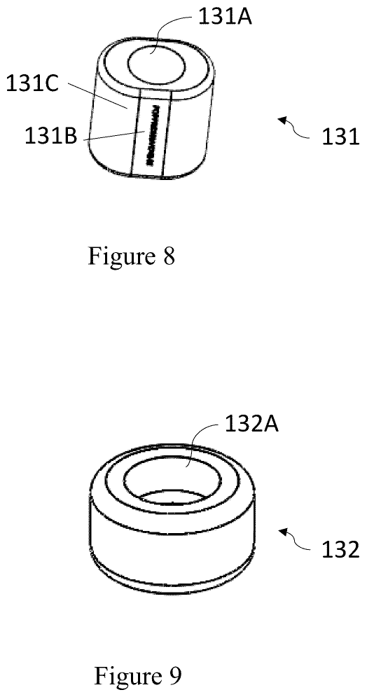

FIG. 8, the string retainer (131) is a cuboid or a cylindroid shaped non-hard component with an axial though-hole (131A) via which the string retainer (131) slides on and tight-fits on the placement tube (114). Once mounted on the placement tube (114), it stay puts linearly as well as rotatingly, that is, it does not rotate nor slide freely. A reference mark (131B) is provided on one of its flat faces (131C) to ascertain its angular position with respect to any mark or projection (144) on the placement tube (114).

FIG. 9, the string lock (132) is a circular non-hard component with a concentric hole (132A) via which the string lock (132) slides on and tight-fits on the placement tube (114). Once mounted on the placement tube (114), it stay puts linearly as well as rotatingly, that is, it does not rotate nor slide freely.

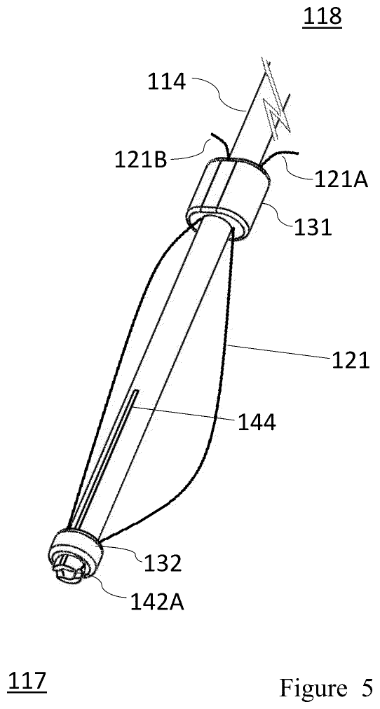

FIG. 5, the instrument (110) is assembled by firstly slipping in the first end (121A) and the second end (121B) of the placement string (121) of the IUD (130) from the fundal end (118) of the placement tube (114), and continuing so with the hanging end(s) (115A) of the retrieval strings (115) also slipped in, and at this point the first end (121A) and the second end (121B) of the placement string (121) would emerge out of the gripping end (117) of the placement tube (114). The first end (121A) and the second end (121B) is then pulled ensuring that the arms (129) of the IUD (130) sit in the two respective constraining slots (142) of the placement tube (114). Keeping both the ends (121A, 121B) of the placement string (121) taut, the string retainer (131) is then slid on the placement tube (114) slipping over the placement string (121), till the string retainer (131) carries the placement string (121) along and is close to the two ends (121A, 121B) of the placement string (121); and the ends of placement string (121) get trapped and constrained consequently. Finally the string lock (132) is slid on the placement tube (114) from the gripping end (117) and positioned just after the gripping end (117) of the placement tube (114). The placement string (121) in the vicinity of the first end (121A) and the second end (121B) is ensured to be sitting in the respective guiding slot (142A) at the gripping end (117) while the placement string (121) is ensured to be taut, such that the arms (129) of the IUD (130) sit firmly in the constraining slots (142) and cannot get dislodged from the constraining slots (142) inadvertently and unless the placement string (121) is loosened consciously or unlocked from string lock (132) as well as string retainer (131).

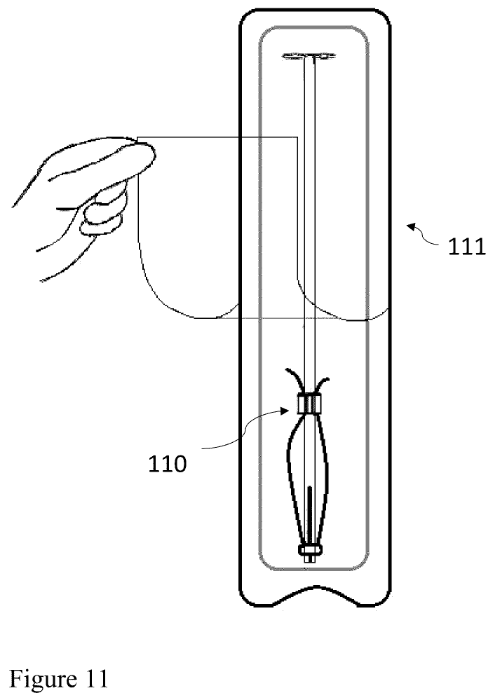

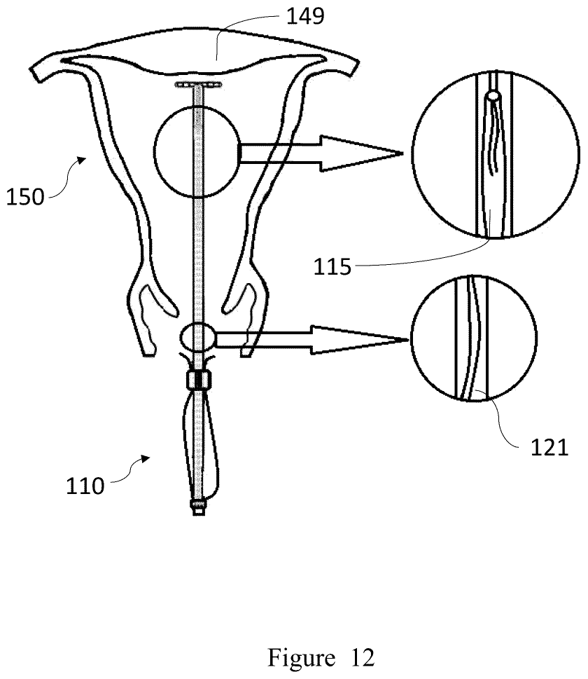

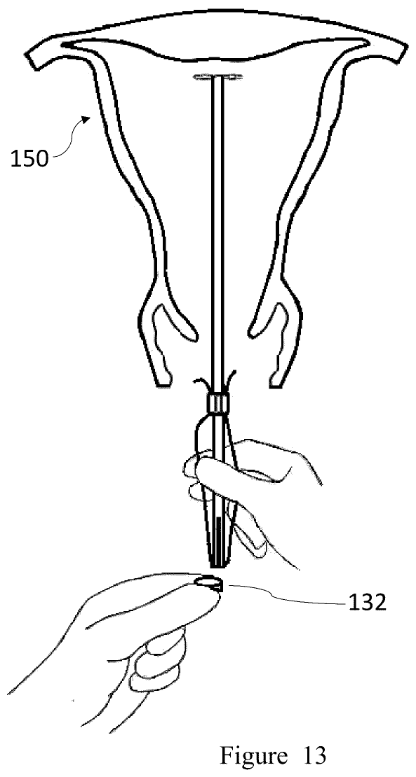

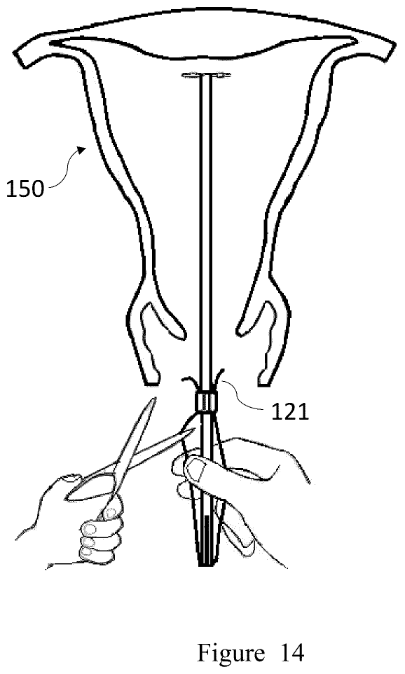

FIG. 11-14, for process of inserting, the instrument (110) is taken out of this transparent pouch (111) by holding the placement tube (114) from the gripping end (117). The instrument (110) is inserted into uterus (150) from the fundal end (118), by continuing to hold the placement tube (114) with one palm (140). Due to large opening of the dilated vagina, cervix and uterus, the overall size of the IUD (130), and particularly a width (126) of the IUD (130) is insignificant, which is otherwise of high significance in normal i.e. non-postpartum situation. The IUD (130) is carried inside the enlarged uterine cavity till touching of the IUD (130) is sensed by surgeon at the fundus (149) of uterus (150). The specific orientation of the IUD (130), implying maintaining a near parallelity between the frame (128) of the IUD (130) and the rear inner skin surface (148) resting the IUD (130) on the rear inner skin surface (148), is maintained by keeping an eye on the projection (144) and or the string retainer (131).

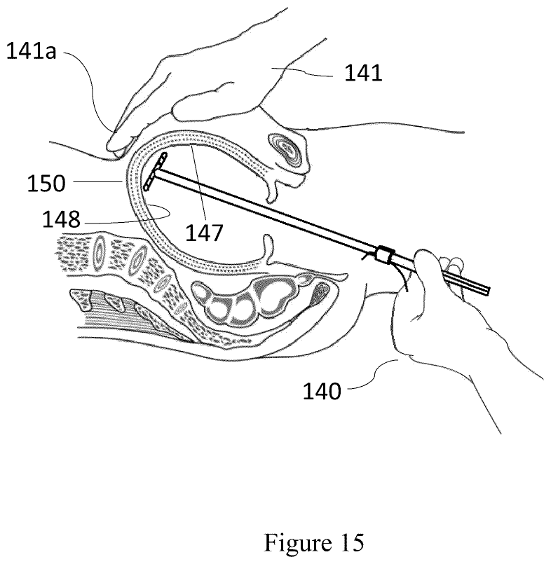

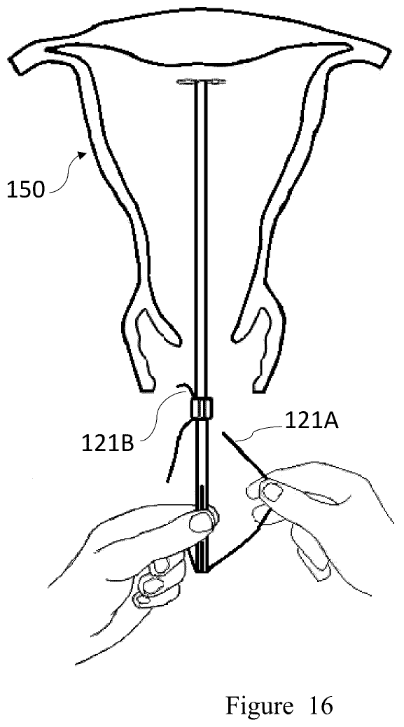

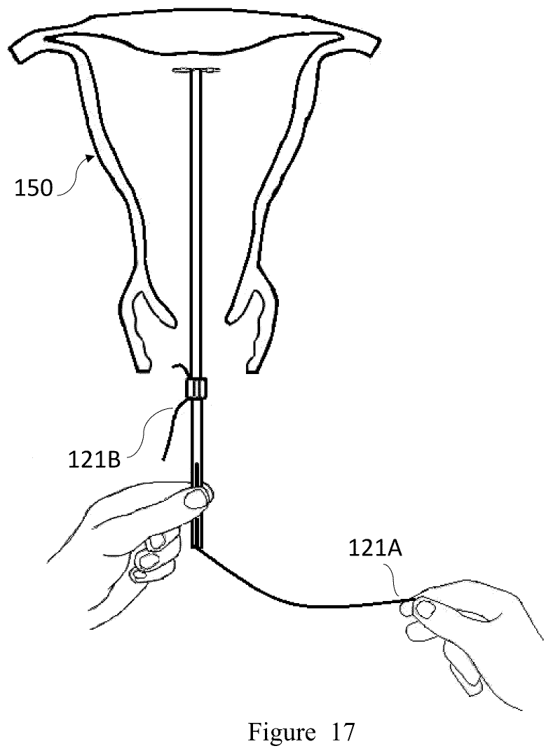

FIG. 15-17, the uterine cavity is felt externally by another palm (141), pushed down externally with palm (141) and the IUD (130) is constrained using any significant finger (141a) on the abdomen. Keeping the IUD (130) constrained externally as thus described, at this moment, the placement string (121) is freed at the gripping end (117) of the placement tube (114) by dismounting the string lock (132) from the gripping end (117). The placement string (121) is cut away by a scissors at a vicinity of the second end (121B) such that a part of the placement string (121) in the vicinity of the second end (121B) remains gripped by the string retainer. The placement string (121) is pulled out from a vicinity of the first end (121A) till the entire remaining placement string (121) gets pulled out of the placement tube (114), including a cut end, created due to cutting of the placement string (121), coming out of the hole of the bulge; and the placement tube (114) is then withdrawn from the uterus (150) gradually and safely.

The function of the retrieval string (115) is twofold:
For a woman to feel its ends (115A) to self-assure presence of her IUD (130) which at times gets dislodged inadvertently.
For retrieving or removing the IUD (130) when woman wishes or at the end of prescribed life of IUD (130).

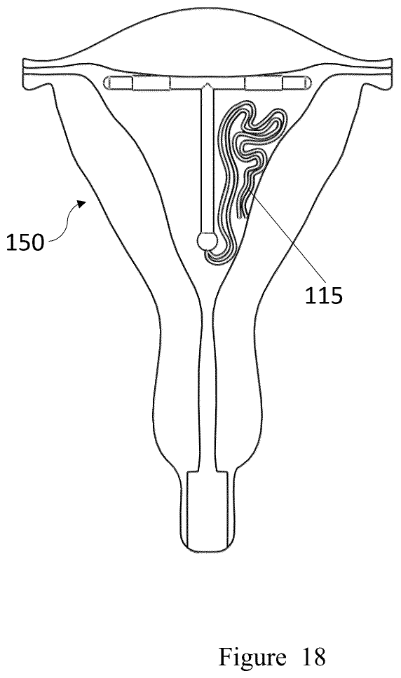

FIG. 18, it is important to note that as the postpartum uterus shrinks gradually, the retrieval string (115) has a tendency to curl and get collected in the uterus randomly. While the function of retrieval or removing is still easily performed by a service provider by pulling the retrieval string (115) out through the cervix, the self-assurance function is lost. FIG. 19, it is due to this reason that the present invention, as a variation, provides for a string of higher rigidity for a part of the retrieval string so that in the course of uterus gradually shrinking, the naturally funnel shaped uterus converging into cervix acts as a mechanical guide and the retrieval string (115) of enhanced rigidity eventually emerges out of the cervical os, as it happens during IUD placement in a normal uterus.

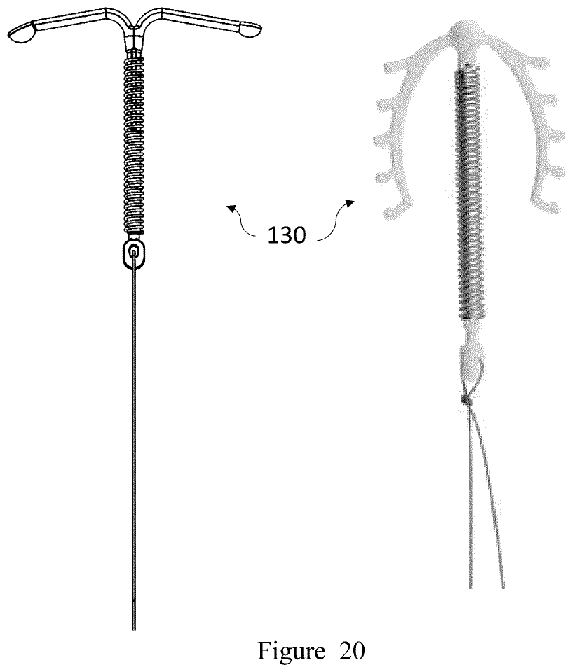

FIG. 20, in above description and corresponding Figures, IUD frame (128) of device (IUD) of commonest shape “T” is shown, however, the instrument and process is suitable for different intra-uterine devices of other shapes. For IUDs of shape other than a “T” frame, any part of construction that prevents rotation of IUD is accordingly constrained by the appropriate constraining means.

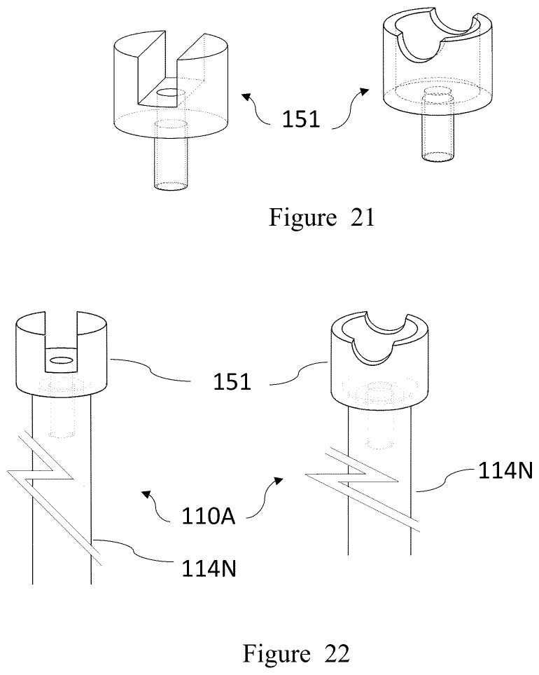

FIG. 21, as a low cost variation, a quick instrument (110A) for postpartum placement of intra-uterine can be prepared in-situ by using a normal insertion tube (114N) wherein the rotation constraining means is an add-on carrier (151) having a pair of constraining slots (142N), equivalently and indirectly disposed at a fundal end of the insertion tube (114N). FIG. 22, such quick instrument (110A) would need higher skills of the service provider.

We claim:

1. An instrument (110) for postpartum placement of intra-uterine device, comprising a tube and an intra-uterine device (IUD), the instrument (110) characterized by:
a placement tube (114) of an extended length (120) having a rotation constraining means at a fundal end (118), a pair of guiding slots (142A) at a gripping end (117), a referencing mark or projection (144) at the gripping end (117) in a defined position with respect to the rotation constraining means,
a placement string (121) disposed removably on the IUD (130) having a retrieval string (115) disposed unremovably,
a string retainer (131) having an axial though-hole (131A), the string retainer (131) firmly stay puts linearly as well as rotatingly on the placement tube (114), and
a string lock (132) having a concentric hole (132A), the string lock (132A) firmly stay puts linearly on the placement tube (114);
the extended length (120) of the placement tube (114) is configured to include a portion of tube length for holding the placement tube (114) from the gripping end (117) after a fundal end of the placement tube (114) touches a fundus (149) of postpartum uterus (150) and a gripping end (117) of the placement tube (114) projects out of vagina of postpartum uterus (150), and
a placement length of the placement string (121) includes a portion of placement string length for holding a first end (121A) of the placement string (121) while a second end (121B) is trapped by the string retainer (131) while the IUD (130) is situated at the fundal end (118) of the placement tube (114) with the placement string (121) taut and arms (129) of the IUD (30) constrained against rotation by the rotation constraining means.

2. The instrument (110) for postpartum placement of intra-uterine device as claimed in claim 1, wherein the rotation constraining means is a pair of constraining slots (142) integrally formed at a fundal end of the placement tube (114).

3. The instrument (110) for postpartum placement of intra-uterine device as claimed in claim 1, wherein the placement tube (114) made of a thermoplastic material is configured to bend axially to adopt to a curvature of postpartum uterus (150).

4. The instrument (110) for postpartum placement of intra-uterine device as claimed in claim 2, wherein the pair of guiding slots (142A) is axially parallel to the pair of constraining slots (142).

5. The instrument (110) for postpartum placement of intra-uterine device as claimed in claim 1, wherein the placement tube (114) has a thickness (119) 0.5 mm to 2.5 mm and a diameter of 5 mm to 8 mm.

6. The instrument (110) for postpartum placement of intra-uterine device as claimed in claim 1, wherein the retrieval string (115) is knotted around a orifice (137) of the bulge (138) from a middle of the retrieval string (115).

7. The instrument (110) for postpartum placement of intra-uterine device as claimed in claim 1, wherein the retrieval string (115) has a hanging length (115B) of 4 cm to 13 cm.

8. The instrument (110) for postpartum placement of intra-uterine device as claimed in claim 1, wherein the retrieval string (115) is integrally molded along with an IUD frame (128) of the IUD (130).

9. The instrument (110) for postpartum placement of intra-uterine device as claimed in claim 8, wherein the retrieval string (115) has a gradually reducing cross section (123) from the bulge towards the hanging end (115).

10. The instrument (110) for postpartum placement of intra-uterine device as claimed in claim 8, wherein the retrieval string (115) has a reducing cross section in at least one steps (124) from the bulge towards the hanging end (115).

11. The instrument (110) for postpartum placement of intra-uterine device as claimed in claim 1, wherein the string retainer (131) has a reference mark (131B) on one of its flat faces (131C).

12. The instrument (110) for postpartum placement of intra-uterine device as claimed in claim 1, wherein the instrument (110) comprises a first end (121A) of the placement string (121) inserted into an orifice (137) of a bulge (138) of the IUD (130), the first end (121A) and the second end (121B) of the placement string (121) of the IUD (130) slipped into placement tube (114) from the fundal end (118), continued with the hanging end(s) (115A) of the retrieval strings (115) slipped in, the first end (121A) and the second end (121B) pulled out from the gripping end (117) while the arms (129) of the IUD (130) sit in the two respective constraining slots (142) of the placement tube (114), keeping both the ends (121A, 121B) of the placement string (121) taut, the string retainer (131) slid on the placement tube (114) slipping over the placement string (121), till the string retainer (131) carries the placement string (121) along and is close to the two ends (121A, 121B) of the placement string(121), the string lock (132) slid on the placement tube (114) from the gripping end (117) and positioned just after the gripping end (117) of the placement tube (114), the placement string (121) in the vicinity of the first end (121A) and the second end (121B) placed in the respective guiding slots (142A) at the gripping end, the placement string (121) being taut.

13. The instrument (110) for postpartum placement of intra-uterine device as claimed in claim 1, wherein the instrument (110) is configured to place the IUD (130) comprising the steps of:
a. taking out the instrument (110) from a sterilized transparent pouch (111),
b. inserting the instrument (110) from the fundal end (118) of the placement tube (114),
c. orienting the placement tube (114) so that the projection (144) is visibly in the middle,
d. dismounting the string lock (132) from the gripping end (117) of the placement tube (114),
e. cutting the placement string (121) at a vicinity of the second end (121B),
f. pulling out the placement string (121) from a vicinity of the first end (121A) till remaining placement string (121) is out of the placement tube, including a cut end, and
g. withdrawing the placement tube (114).

14. A quick instrument (110A) for postpartum placement of intra-uterine device (130), wherein a rotation constraining means is an add-on carrier (151) having a pair of constraining slots (142), the add-on carrier (151) removably disposed at a fundal end of an insertion tube (114N).
